SEQUENCE LISTING (10 sequences)

SEQ ID NO: 1 - protein, 517 aa, Homo sapiens
MARMGLAGAAGRWWGLALGLTAFFLPGVHSQVVQVNDSMYGFIGTDVVLHCSFANPLPSVKITQVTWQKSTNGSKQNVAIYNPSMGVSVLAPYRERVEFLRPSFTDGTIRLSRLELEDEGVYICEFATFPTGNRESQLNLTVMAKPTNWIEGTQAVLRAKKGQDDKVLVATCTSANGKPPSVVSWETRLKGEAEYQEIRNPNGTVTVISRYRLVPSREAHQQSLACIVNYHMDRFKESLTLNVQYEPEVTIEGFDGNWYLQRMDVKLTCKADANPPATEYHWTTLNGSLPKGVEAQNRTLFFKGPINYSLAGTYICEATNPIGTRSGQVEVNITEFPYTPSPPEHGRRAGPVPTAIIGGVAGSILLVLIVVGGIVVALRRRRHTFKGDYSTKKHVYGNGYSKAGIPQHHPPMAQNLQYPDDSDDEKKAGPLGGSSYEEEEEEEEGGGGGERKVGGPHPKYDEDAKRPYFTVDEAEARQDGYGDRTLGYQYDPEQLDLAENMVSQNDGSFISKKEWYV

SEQ ID NO: 2 - protein, 34 aa, Homo sapiens
GPINYSLAGTYICEATNPIGTRSGQVEVNITEFP

SEQ ID NO: 3 - protein, 517 aa, Homo sapiens
MARMGLAGAAGRWWGLALGLTAFFLPGVHSQVVQVNDSMYGFIGTDVVLHCSFANPLPSVKITQVTWQKSTNGSKQNVAIYNPSMGVSVLAPYRERVEFLRPSFTDGTIRLSRLELEDEGVYICEFATFPTGNRESQLNLTVMAKPTNWIEGTQAVLRAKKGQDDKVLVATCTSANGKPPSVVSWETRLKGEAEYQEIRNPNGTVTVISRYRLVPSREAHQQSLACIVNYHMDRFKESLTLNVQYEPEVTIEGFDGNWYLQRMDVKLTCKADANPPATEYHWTTLNGSLPKGVEAQNRTLFFKGPINYSLAGAYICEATNPIGTRSGQVEVNITEFPYTPSPPEHGRRAGPVPTAIIGGVAGSILLVLIVVGGIVVALRRRRHTFKGDYSTKKHVYGNGYSKAGIPQHHPPMAQNLQYPDDSDDEKKAGPLGGSSYEEEEEEEEGGGGGERKVGGPHPKYDEDAKRPYFTVDEAEARQDGYGDRTLGYQYDPEQLDLAENMVSQNDGSFISKKEWYV

SEQ ID NO: 4 - protein, 34 aa, Homo sapiens
GPINYSLAGAYICEATNPIGTRSGQVEVNITEFP

SEQ ID NO: 5 - protein, 517 aa, Homo sapiens
MARMGLAGAAGRWWGLALGLTAFFLPGVHSQVVQVNDSMYGFIGTDVVLHCSFANPLPSVKITQVTWQKSTNGSKQNVAIYNPSMGVSVLAPYRERVEFLRPSFTDGTIRLSRLELEDEGVYICEFATFPTGNRESQLNLTVMAKPTNWIEGTQAVLRAKKGQDDKVLVATCTSANGKPPSVVSWETRLKGEAEYQEIRNPNGTVTVISRYRLVPSREAHQQSLACIVNYHMDRFKESLTLNVQYEPEVTIEGFDGNWYLQRMDVKLTCKADANPPATEYHWTTLNGSLPKGVEAQNRTLFFKGPINYSLAGTAICEATNPIGTRSGQVEVNITEFPYTPSPPEHGRRAGPVPTAIIGGVAGSILLVLIVVGGIVVALRRRRHTFKGDYSTKKHVYGNGYSKAGIPQHHPPMAQNLQYPDDSDDEKKAGPLGGSSYEEEEEEEEGGGGGERKVGGPHPKYDEDAKRPYFTVDEAEARQDGYGDRTLGYQYDPEQLDLAENMVSQNDGSFISKKEWYV

SEQ ID NO: 6 - protein, 34 aa, Homo sapiens
GPINYSLAGTAICEATNPIGTRSGQVEVNITEFP

SEQ ID NO: 7 - protein, 517 aa, Homo sapiens
MARMGLAGAAGRWWGLALGLTAFFLPGVHSQVVQVNDSMYGFIGTDVVLHCSFANPLPSVKITQVTWQKSTNGSKQNVAIYNPSMGVSVLAPYRERVEFLRPSFTDGTIRLSRLELEDEGVYICEFATFPTGNRESQLNLTVMAKPTNWIEGTQAVLRAKKGQDDKVLVATCTSANGKPPSVVSWETRLKGEAEYQEIRNPNGTVTVISRYRLVPSREAHQQSLACIVNYHMDRFKESLTLNVQYEPEVTIEGFDGNWYLQRMDVKLTCKADANPPATEYHWTTLNGSLPKGVEAQNRTLFFKGPINYSLAGAAICEATNPIGTRSGQVEVNITEFPYTPSPPEHGRRAGPVPTAIIGGVAGSILLVLIVVGGIVVALRRRRHTFKGDYSTKKHVYGNGYSKAGIPQHHPPMAQNLQYPDDSDDEKKAGPLGGSSYEEEEEEEEGGGGGERKVGGPHPKYDEDAKRPYFTVDEAEARQDGYGDRTLGYQYDPEQLDLAENMVSQNDGSFISKKEWYV

SEQ ID NO: 8 - protein, 34 aa, Homo sapiens
GPINYSLAGAAICEATNPIGTRSGQVEVNITEFP

SEQ ID NO: 9 - protein, 517 aa, Homo sapiens
MARMGLAGAAGRWWGLALGLTAFFLPGVHSQVVQVNDSMYGFIGTDVVLHCSFANPLPSVKITQVTWQKSTNGSKQNVAIYNPSMGVSVLAPYRERVEFLRPSFTDGTIRLSRLELEDEGVYICEFATFPTGNRESQLNLTVMAKPTNWIEGTQAVLRAKKGQDDKVLVATCTSANGKPPSVVSWETRLKGEAEYQEIRNPNGTVTVISRYRLVPSREAHQQSLACIVNYHMDRFKESLTLNVQYEPEVTIEGFDGNWYLQRMDVKLTCKADANPPATEYHWTTLNGSLPKGVEAQNRTLFFKGPINYSLAGTYICEATNPIGTRAGQVEVNITEFPYTPSPPEHGRRAGPVPTAIIGGVAGSILLVLIVVGGIVVALRRRRHTFKGDYSTKKHVYGNGYSKAGIPQHHPPMAQNLQYPDDSDDEKKAGPLGGSSYEEEEEEEEGGGGGERKVGGPHPKYDEDAKRPYFTVDEAEARQDGYGDRTLGYQYDPEQLDLAENMVSQNDGSFISKKEWYV

SEQ ID NO: 10 - protein, 34 aa, Homo sapiens
GPINYSLAGTYICEATNPIGTRAGQVEVNITEFP

(19) United States
(12) Patent Application Publication
(10) Pub. No.: US 2010/0285597 A1
(43) Pub. Date: Nov. 11, 2010

(54) DETECTION OF FRAGMENTS OF NECTIN-1 FOR THE DIAGNOSIS OF ALZHEIMER'S DISEASE

(75) Inventors: Howard Federoff, Rochester, NY (US); Seung T. Lim, Rochester, NY (US)

Correspondence Address: BLANK ROME LLP, WATERGATE, 600 NEW HAMPSHIRE AVENUE, N.W., WASHINGTON, DC, 20037, US

(73) Assignee: UNIVERSITY OF ROCHESTER, ROCHESTER, NY (US)

(21) Appl. No.: 12/601,673

(22) PCT Filed: May 27, 2008
(86) PCT No.: PCT/US08/64899

§ 371 Date: Jul. 16, 2010

Related U.S. Application Data
(60) Provisional application No. 60/924,661, filed on May 24, 2007.

(51) Int. Cl. G01N 33/68; C07H 21/04; C07K 14/47; G01N 33/559

(52) U.S. Cl. 436/89; 536/23.5; 530/350; 204/461

(57) ABSTRACT

Methods for diagnosing Alzheimer's Disease by detection of fragments of nectin-1 are described. Nectin-1 is shown to be a substrate for proteases associated with the onset of Alzheimer's Disease, including α-secretase, γ-secretase and BACE1 or a BACE1-like protease. The fragments produced by the action of these and other proteases on Nectin-1 can be used to diagnosis Alzheimer's Disease or a predilection towards Alzheimer's Disease.

### STATEMENT OF GOVERNMENT INTEREST

This invention was made with U.S. Government support by the National Institutes of Health and is assigned NIH Grant No. 5R01AG27233. The government has certain rights in the invention.

### FIELD OF THE INVENTION

The present invention relates to the detection and diagnosis of Alzheimer's disease.

### BACKGROUND

The number of Americans with Alzheimer's disease (AD) continues to grow as the population ages. Approximately one in ten people over the age of sixty five, and five in ten people over the age of 85 have some form of the disease. This trend will continue as life expectancies continue to increase. Currently, there are approximately 4.5 million Americans affected by the disease. It is estimated that this number will grow to be between 11 and 16 million by 2050.

While certain molecular mechanisms related to AD formation have become better understood in the past few years, the exact causative mechanism of the disease is still unknown. Although there are several theories on the initiating events in AD formation, there are two main mechanistic events that are known to occur in conjunction with progression of the disease. One of these events is the formation of neurofibrilary tangles caused by the hyperphosphorylation of tau, a micro-tubule binding protein. The other is the formation of plaques consisting of amyloid beta (Aβ) peptides formed from cleavage of amyloid precursor protein (APP).

Both neurofibrilary tangles and Aβ plaques are found in failing synapses during AD formation. Neurofibrilary tangles form inside nerve cells while Aβ plaques form extracellularly, disrupting synaptic connections. As synapses fail, neuronal death occurs, leading to wasting of the brain and increased onset of AD symptoms.

Other lines of evidence have implicated the formation of Aβ with the onset of AD. The most compelling of this evidence is that several mutations associated with early-onset familial AD are all linked with abnormal processing of APP and Aβ. This includes mutations in the presenilin 1 and 2 genes, which are involved in the cleavage of APP, and mutations in APP itself. Furthermore, it has been shown that mice overexpressing APP develop AD-like symptoms (Hsiao et al., 1996; Hsia et al., 1999).

Aβ is formed when a series of proteases called secretases act on APP. Membrane bound APP can be cleaved by one of two secretase pathways. APP can be cleaved by α-secretase followed by one of the γ-secretases to form the soluble and nonamyloidogenic peptide p3. Alternatively, APP can be cleaved by β-secretase followed by a γ-secretase cleavage that forms Aβ peptides of various lengths, primarily of 40 (Aβ40) and 42 (Aβ42) amino acids. Both of the Aβ peptides are found in amyloid plaques. However, it has been shown that Aβ42 causes formation of the plaques more rapidly.

Synaptic disfunction and failure are processes that occur very early in the development of AD. These changes in synaptic pathology can begin to occur decades before the onset of clinical symptoms of AD (Coleman ref.). In one study, nearly 20% of persons autopsied who had died in their 20s showed the synaptic pathology of AD (Braak ref). By contrast, patients are typically in their mid-70s before clinical symptoms of AD begin to manifest. As such, detection of changes in synapse structure and function could be used to diagnose a patient with AD 50 years before they begin to show symptoms of the disease.

The early detection of AD could lead to better treatment and management of symptoms. Currently, there is no reliable method for pre-mortem physiological detection of AD, and most speculated cases are confirmed only upon autopsy. Although clinical diagnosis of AD has become more and more accurate, this diagnosis is only possible once AD symptoms have become prevalent in a patient, and does not allow for any pre-emptive therapy. One method that is being developed in the detection of biomarkers from human fluids and tissues to determine the onset and progression of AD. Changes in levels of a specific biomarker or a set of biomarkers can indicate the onset and progression of AD. Sets of biomarkers are being developed, but there continues to be a need in the art for further and more reliable biomarkers for the detection of AD.

### SUMMARY OF THE INVENTION

From the above, it should be apparent that the need exists for improved methods for the diagnosis of AD. It is an object of the present invention to provide improved methods for diagnosing AD through the detection of fragments of nectin-1.

The methods of diagnosis of AD may include detection of one or more fragments of nectin-1 as produced by different proteases. The fragments detected in the present invention may include fragments which are the products of the cleavage of nectin-1 by α secretase, γ secretase, BACE-1, or proteases with similar activities and cleavage site specificities for nectin-1.

It is a an object of the present invention to provide a method for diagnosing AD using a Herpes Simplex Virus (HSV) based system. It has been shown that the soluble form of HSV glycoprotein-D binds to Nectin-1 and causes its proteolysis. The fragment generated by this proteolysis could serve as a biomarker for AD.

It is a further object of the invention to provide a method for developing a biomarker database for the detection of AD. Samples from patients with or without clinical symptoms of AD may be analyzed for the presence of nectin-1 fragments. The patterns of fragments detected in the patient may then be correlated in a database with the clinical diagnosis of the patient. After a database is assembled, it can be used to compare the fragment patterns in the database with the fragment patterns of a new patient sample, wherein when a patient has fragment pattern that correlates with a diagnosis of AD in the database, the patient is diagnosed as having AD or a predilection to AD.

### BRIEF DESCRIPTION OF THE FIGURES

FIG. 1(A) shows a Western blot from rat brain membrane, cultured hippocampal neurons, and primary astrocytes incubated with nectin-1 antibody.

FIG. 1(B-E) shows immunofluorescence staining to localize nectin-1. Panels are further described in Example 1.

FIG. 2(A-D) shows a Western blot demonstrating the ectodomain shedding of nectin-1. Panels and lanes are as described in Example 2.

FIG. 3 is a Western blot showing the overexpression of nectin-1 in hippocampal neurons. Lanes of the blot are as described in Example 3.

FIG. 4(A-B) is a Western blot of an anti-BACE1 immunoprecipitation from crude rat brain. Panels are as described in Example 4.

FIG. 5(A-D) shows the subcellular distribution of BACE1 in hippocampal neurons. Panels are as described in Example 5.

FIG. 6 is a Western blot demonstrating that β-secretase inhibitor blocks cleavage of nectin-1. Lanes of the blot are as described in Example 6.

FIG. 7 is a schematic diagram of the nectin-1 ectodomain deletion mutants used to define the minimal region that contains the β cleavage site. A panel of progressive external truncation mutants was generated by inverse PCR mutagenesis. V5 and Flag tags were inserted at the N-terminus right after the signal sequence and the C-terminus, respectively (see Example 7).

FIG. 8 shows immunofluorescence staining of the nectin-1 truncation mutants. Panels are as described in Example 7.

FIG. 9 is a Western blot of a series of ectodomain truncation mutant proteins. Lanes are as described in Example 8.

FIG. 10(A-D) is a Western blot of an immunoprecipitation done on a series of ectodomain truncation mutant proteins. Lanes and panels are as described in Example 9.

FIG. 11 is a Western blot of an analysis of nectin-1 point mutations from amino acids 301-334. Lanes are as described in Example 10.

FIG. 12(A-D) is a Western blot of an immunoprecipitation done on a series of nectin-1 point mutants. Lanes and panels are as described in Example 11.

FIG. 13(A-D) shows the cellular localization of nectin-1 and nectin-1 point mutants in COS-7 cells. Panels are as described in Example 12.

FIG. 14(A-F) shows the cellular co-localization of nectin-1 and nectin-1 point mutants with BACE1 in COS-7 cells. Panels are as described in Example 13.

FIG. 15 is a Western blot demonstrating the nectin-1 point mutants T310A and Y311A interfere with the processing of wildtype nectin-1. Panels are as described in Example 13.

### DETAILED DESCRIPTION OF THE INVENTION

For the first time, it is reported here that nectin-1 is cleaved by BACE1 or a BACE-1 like protease in a process analogous to the processing of APP (See examples below). Further, for the first time, it is reported here that nectin-1 undergoes ectodomain shedding by α secretase and subsequent processing by γ secreatase in a process regulated by Ca²⁺/calmodulin.

### Nectin-1

Nectins are members of the immunoglobulin (Ig) like cell-cell adhesion molecule and are involved in cell-cell adherens junctions (AJs) (Takai and Nakanishi, 2003). The nectin family consists of four members, nectin-1, 2, 3, and 4. Nectins 1, 2 and 3 have splicing variants nectin-1α, -1β, -1γ, -1δ; nectin-2α, 2δ; and nectin-3α, -3β, -3γ (Takai et al. 2003, Takai and Nakanishi, 2003). All nectins, except nectin-1γ, have one extracellular region composed of three Ig-like loops, one transmembrane domain, and one cytoplasmic tail (Lopez et al. 1995). Nectin-1γ lacks a transmembrane and cytoplasmic domain.

Nectins, through interaction with afadin, connect to the actin-based cytoskeleton (Mandai et al., 1997). Most nectins contain a type II PDZ binding motif (Glu/Ala-X-tyr-Val) on their C-terminus that interacts with the PDZ domain of afadin. Each member of the nectin family can form a homo-cis-dimer and subsequent home-trans-dimer, thereby promoting cell-cell adhesion. Further, nectin-3 can form a hetero-trans-dimer with either nectin-1 or nectin-2, while nectin-4 may hetero-trans-dimerize with nectin-1 (Takai et al., 2003). In general, the hetero-trans-dimers interactions are stronger than the homo-trans-dimers. The first Ig-like domain of nectin participates in cis- and trans-dimerization, while the second Ig-like domain is necessary for trans-dimerization.

### The Role of Nectins in Organization of the Epithelial/Fibroblast Junctional Complex

Nectins form cell-cell contacts and recruit cadherens to form adherins junctions (AJs) (Tachibana et al., 2000; Honda et al., 2003). Nectin-1 also recruits ZO-1 through afadin in a cadherin-independent manner in fibroblasts (Yokoyama et al., 2001). Nectins induce activation of Cdc42 and Rae, small G proteins (Kawakatsu et al, 2002; Honda et al., 2003). Activated Cdc42 in turn induces filopodia and increases the number of cell-cell contacts sites at the initial stage of AJ formation. Activated Rac small G molecules stimulate lamellipodia, thus efficiently expanding the cell-cell adhesion between filopodia. In epithelial cells, nectins recruit the initial junctional adhesion molecules and then claudins to the apical side of AJs, resulting in assembly of tight junctions (TJs). The cell polarity complex of Par-3, atypical protein kinase C, and Par-6 is essential for the formation of TJs, and Cdc42 induces the activation of this complex by binding Par-6 (Ohno, 2001). Nectins directly interact with Par-3, suggesting that cdc42, once activated by the action of nectin-1, helps mediate activation of the cell polarity protein complex (Takekuni et al., 2003). Recently, it has been shown that nectins recruit c-Src to nectin-based cell-cell contact sites and that c-Src activates Rapl through the Crk-C3G complex (Fukuyama et al., 2005). Activated Rapl activates tyrosine-phosphorylated FRG, a Cdc42-GDP/GTP exchange factor, followed by activation of Cdc42 itself (Fukuhara et al., 2004; Fukuyama et al., 2005). These data indicate that nectins play an essential role in the formation of cell-cell junctions and cell polarity through Cdc42 and Rac.

### The Role of Nectins in Organization of Synapses

Synapses are specialized intercellular junctions that are formed when a presynaptic terminal contacts a postsynaptic neuron. Neurotransmission depends on synaptic specificity and acquisition of new information depends on the ability to remodel synapses. Nectin-1 mRNAs are detected in the human CNS (Cocchi et al., 1998), in neuronal cell lines (Geraghty et al., 1998), and in mouse sensory, sympathetic and parasympathetic neurons (Haarr et al., 2001; Richart et al., 2003). Data presented in the Examples shows that, in the CNS, nectin-1 is found in both neurons and astrocytes. Nectin co-localizes with afadin at the synapse (Mizoguchi et al., 2002). Nectin-1 and -3 localize at the pre- and post-synaptic sides of puncta adherentia junctions (PAJs) formed in the CA3 pyramidal region of the adult mouse hippocampus, respectively. The addition of a nectin-1 and -3 inhibitor to cultured rat hippocampal neurons alters the cellular distribution of synaptophysin and PSD-95 and decreases the size but increases the number of synapses (Mizoguchi et al., 2002). Furthermore, the nectin-1/afadin system associates with the N-cadherin-catenin system in early synaptogenesis in cultured rat hippocampal neurons. Mutations in the nectin-1 gene cause cleft lip/palate-ectodermal dysplasia and, in some cases, mental retardation (Suzuki et al., 2000; Sozen et al., 2001). These data suggest that nectin plays an important role in synaptogensis.

### Ectodomain Shedding of Nectin-1 and its Possible Roles

Nectin-1 undergoes ectodomain shedding upon treatment with SFIHGF or TPA in MDCK cells (Tanaka et al., 2002) and in CHO cells (Kim et al., 2002), generating a large soluble fragment and small C-terminal fragment (CTF). As shown in the Examples, the shedding of nectin-1 also occurs at synapses in mature hippocampal neurons. Nectin-1 ectodomain shedding is inhibited by metalloprotease inhibitors (Tanaka et al., 2002) suggesting that a metalloprotease may be involved in the process.

There at least three possible physiological functions of ectodomain shedding. First, it causes disruption of cell AJs resulting in loss of cell-cell contact in fibroblast and epithelial cells. Nectin plays an essential role in the organization of the junctional complex comprised of E-cadherin based AJs and claudin-based TJs (Asakura et al., 1999; Takahashi et al., 2003; Fukuhara et al., 2002; Honda et al., 2003; Hoshino et al., 2003; Katata et al., 2003). It has also been shown that E-cadherin undergoes ectodomain shedding mediated by a metalloprotease. Overall, these data suggest that cell-cell disassociation requires shedding of both cell adhesion molecules by common sheddases.

A possible second physiological function of ectodomain shedding of nectin-1 is that the soluble fragment of nectin-1 released during the process may elicit a biological response by binding to nectin-1 or -3 or to an unknown ligand. Experiments have shown that a fusion protein composed of the ectodomain of nectin-1 and the Fc portion of IgG trans-interacts with cellular nectin-1 and -3 and induces filopodia and lamellipodia through the sequential activation of Cdc42 and Rac (Kawakatsu et al., 2002; Honda et al., 2003).

A possible third physiological function of ectodomain shedding of nectin-1 is that, in neurons, nectin-1 may regulate synapse formation and remodeling. Cells control synaptic plasticity by regulating density of cell adhesion molecules such as nectin-1 or other cell adhesion molecules. These structural modifications of synapses are likely the underlying cause of synapse plasticity implicated in learning and memory.

### β-Secretase and Nectin-1

BACE1 has been identified as the β-secretase that cleaves APP within the ectodomain (Hussain et al., 1999; Sinha et al., 1999; Vassar et al., 1999; Yan et al., 1999). BACE1 displays some homology to the pepsin family of aspartyl proteases and is described in U.S. Pat. No. 6,727,074 which is hereby incorporated by reference herein. BACE1 is ubiquitously expressed with its highest level of expression found in neurons. BACE1 is synthesized in the ER as a preproprotein, then processed to its mature form in the Golgi compartment by furin-like convertases (Bennett et al, 2000; Capell et al., 2000; Benjannet et al., 2001; Pinnix et al., 2001). Although BACE1 has enzymatic activity in the ER compartment, endogenous mature BACE1 predominantly localizes in the trans-Golgi network, where it cleaves APP to produce a secreted N-terminal fragment and a C-terminal membrane bound fragment (Yan et al., 2001). The C-terminal fragment is subsequently processed by γ-secretase to release amyloidogenic Aβ peptides, predominantly Aβ40 and Aβ42 (Luo et al., 2001; Roberds et al., 2001).

Mice deficient in BACE1 are healthy and fertile and have a near complete absence of Aβ (Luo et al., 2001; Roberds et al., 2001). Mice lacking BACE1 exhibit an anxious and less exploratory behavior (Harrison et al., 2003), suggesting that BACE1 is involved in synaptic neurotransmission.

It is presented in the Examples, for the first time, that BACE1 cleaves the ectodomain of nectin-1, generating a 37 kDa C-terminal fragment. Treatment with BACE1 inhibitor establishes that residues 301 to 333 in the ectodomain of nectin-1 are necessary for association with BACE1. Furthermore, BACE1 colocalizes with nectin-1 at the synapses and associates with nectin-1 in the brain. These data suggest a role for BACE1 as a synaptic modulator.

### Nectin-1 and Alzheimer's Disease

As mentioned, in AD, synapse loss is an early event in the development of the disease and is a structural correlate of cognitive dysfunction. APP transgenic mice exhibit abnormalities in learning/memory and synaptic function but the molecular mechanisms by which increased Aβ levels affect these functions remain undefined (Hsiao et al., 1996; Hsia et al., 1999).

Interestingly, nectin-1 undergoes proteolytic processing in a manner analogous to APP, mediated first by BACE1 (as shown in the Examples) followed by cleavage with a presenilin dependent γ-secretase (Kim et al., 2002). This observation is consistent with a previously reported role for PS/γ-secretase in AJ function involving cadherin cleavage (Baki et al., 2001; Marambaud et al., 2002; Marambaud et al., 2003). The role of nectin-1 in synapse formation and remodeling raises the possibility that familial Alzheimer's disease mutations in presenilin1 may directly perturb synaptic activity by aberrantly modulating nectin processing and producing synaptic dysfunction.

### NMDA Receptor Activation and Nectin-1 Cleavage

Activation of NMDA receptors causes an increase in Ca²⁺ concentration in the post-synaptic neuron, triggering induction of protein kinase pathways that lead to long term potentiation (LTP) of the synapse. LTP is an increase in the strength of the synapse that can last from several minutes to several days and has been hypothesized to play a critical role in synaptic plasticity and memory formation (see Kandel et al., Principles of Neuroscience, 4^{th} ed., Chapter 63). Because of the link between LTP and memory formation, that treatment of AD may be possible through the manipulation of this pathway.

It is shown in the Examples below, for the first time, that nectin-1 undergoes processing by the α and γ secretases in a Ca²⁺/calmodulin dependent manner upon activation of NMDA receptors. These results show that this type of nectin-1 processing is occurring concurrently with events involved in increasing synaptic plasticity and/or LTP.

### Diagnosis of AD

In a preferred embodiment of the invention, nectin-1 fragments are produced and detected by infecting a patient or a sample taken from a patient with a non-innoculous strain of Herpes Simplex Virus (HSV). The soluble HSV protein glycoprotein-D binds nectin-1 and causes its proteolysis (Geraghty et al., 1998). The fragments of nectin-1 produced by this process could be detected and correlated to a diagnosis of AD.

As described herein, activation of NMDA receptors causes cleavage of nectin-1 by the α and γ secretases. Because activation of NMDA receptors is associated with LTP, memory formation and synaptic plasticity, detection of the nectin-1 fragments formed after processing with a or γ secretase could be correlated to events occurring at the synapses. As a non-limiting example, upon introduction of HSV to a patient or sample, the fragments of nectin-1 formed could be analyzed to determine if nectin-1 had been cleaved by α secretase. The presence of a large number of α secretase cleaved nectin-1 fragments could indicate high NMDA receptor activity in the patient, suggesting that events such as LTP were actively occurring. Comparisons can be made of the same patient or samples from a patient over time, with a decrease in α secretase fragments suggesting decreasing NMDA receptor activation, decreased LTP and the possible onset of, or predilection to, AD. The same type of methodology could also be applied to fragments of nectin-1 generated by secretase or by processing by both α and γ secretases.

In another embodiment of the present invention the diagnosis of AD is performed by detection of nectin-1 fragments produced by BACE1 or BACE1-like cleavage. A BACE-1 like cleavage is the action of a protease with similar features to BACE-1, such a protease that cleaves nectin-1 at the same or similar cleavage site, either by sequence or structure recognition. As BACE1 activity is involved in the pathogenesis of AD, detection of fragments of nectin-1 produced BACE1 cleavage could be used to diagnose and determine the progression of AD. An increase in the number of nectin-1 cleavage fragments can be associated with an increase in BACE1 activity, suggesting the onset of, or a patient's predilection to, AD.

The invention also contemplates the detection of fragments produced by proteases, other enzymes, or chemical or physical methods, and is not meant to be limited to cleavage of nectin-1 by α secretase, γ secretase, BACE-1 or a BACE-1 like protease. The other methods for cleaving nectin-1 contemplated by the invention may or may not cleave nectin-1 in the same part of the protein as the enzymes described herein.

In a preferred embodiment of the invention, fragments of nectin-1 are detected by methods of polypeptide detection well known in the art, such as mass spectrometry or two-dimensional gel electrophoresis. For non-limiting examples of detection methods, see Zhou et al. (2005) and Davidsson et al. (2005), which are hereby incorporated by reference herein.

In another embodiment of the invention, nectin-1 fragments are detected by an antibody to nectin-1. The antibody may be able to detect specific fractions of nectin-1, and, in a preferred embodiment, the antibody is able to detect a specific fragment of nectin-1 that is produced after cleavage with one or more of α secretase, γ secretase, BACE1 or a BACE1-like enzyme. In a more preferred embodiment, the antibody is able to detect the nectin-1 fragment but not full length nectin-1, due to the fact that the epitope which the antibody recognizes is masked or hidden in the full length protein. Preferable detection methods using nectin-1 antibodies include detection by Western blot or Enzyme-Linked Immunosorbent Assay (ELISA).

For detection of nectin-1 fragments in a patient, samples must be obtained from the patient. In a preferred embodiment, the samples obtained from the patient are bodily fluids, for example, blood, urine, saliva and cerebrospinal fluid (CSF). Other samples obtained may be of solid tissues obtained through biopsy or similar methods.

Correlations of the type and concentration of nectin-1 fragments with the onset of or predilection to AD may be made in various ways. One possibility for correlating the presence of nectin-1 fragments with AD is to monitor the level of a specific fragment or fragments over time in the same patient or sample. An increase or decrease of a fragment over time may then be correlated with changes in the synapse, LTP and/or potential onset of AD.

Another method for correlating the nectin-1 fragments detected in a patient or sample with an AD diagnosis is through the use of a database of fragments of nectin-1 correlated with clinical or other symptoms of AD. A biomarker database may be assembled by analyzing the nectin-1 fragments present in various populations of subjects, such as those with early or late stage AD, subjects with a family history of AD and subjects with no signs of AD. Correlations between disease symptoms and various nectin-1 fragments may then be made using methods known in the art. Examples of methods for diagnosis of AD using different biomarkers include Blennow and Hampel, Lancet Neurology 2:605-13, 2003 and Blennow, J. of Internal Medicine 256:224-34, 2004, which are hereby incorporated by reference herein.

### Mutations of Nectin-1

The amino acid sequence of human wild type nectin-1 is represented by SEQ ID NO:1. SEQ ID NO:2 is a polypeptide representing the BACE1 interacting domain of nectin-1 as defined in Example 9 below. SEQ ID NO:3 represents the T310A point mutant of nectin-1 that shows enhanced binding to BACE1. SEQ ID NO:4 represents the BACE1 interacting domain of nectin-1 containing the T310A point mutation. SEQ ID NO:5 represents the Y311A point mutant of nectin-1 that shows enhanced binding to BACE1. SEQ ID NO:6 represents the BACE1 interacting domain of nectin-1 containing the Y311A point mutation. SEQ ID NO:7 represents the T310A Y311A double mutant of nectin-1. SEQ ID NO:8 represents the BACE1 interacting domain of nectin-1 containing the T310A Y311A double mutation. SEQ ID NO:9 represents the S323A point mutant of nectin-1 that shows enhanced binding to BACE1. SEQ ID NO:10 represents the BACE1 interacting domain of nectin-1 containing the S323A point mutation.

### Example 1

Nectin-1 is expressed in hippocampal neurons and localized to the excitatory synapses.

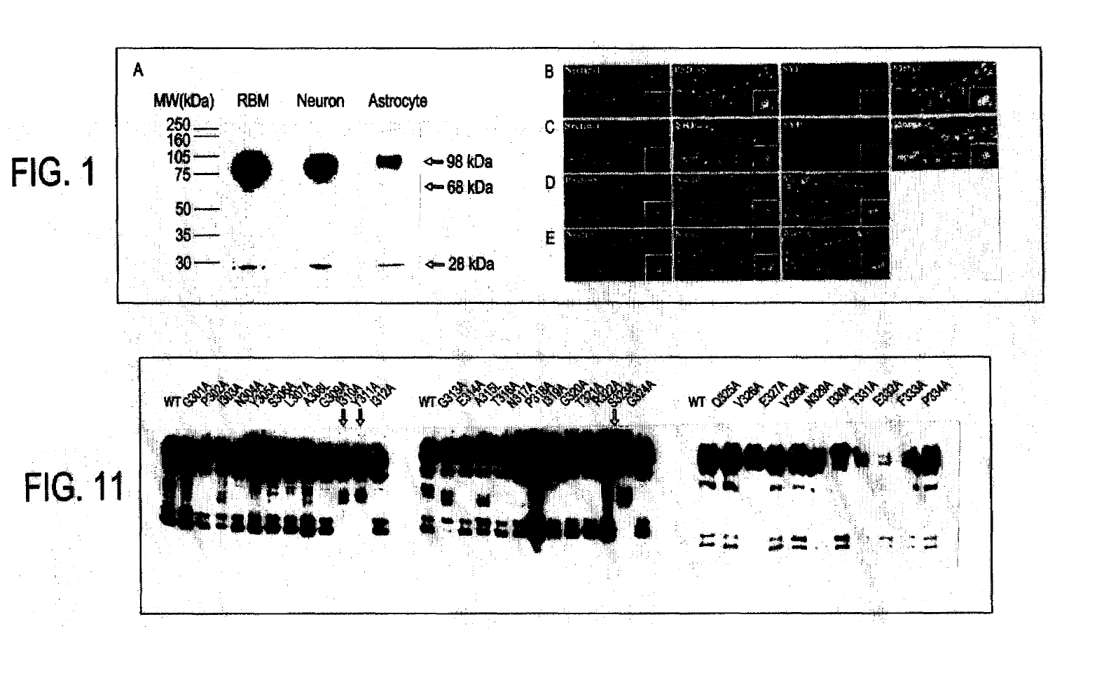

FIG. 1A shows a Western blot of lysates from rat brain membrane, DIV14 cultured hippocampal neurons, and primary astrocytes, incubated with polyclonal antibody against nectin-1. Three distinct bands are shown (98, 68 and 28 kDa). Neurons at 28 DIV were methanol fixed and labeled with synaptic markers. In FIG. 1B, neurons were triple stained with nectin-1 (red), PSD-95 (green) and synaptophysin (blue). Immunofluorescence images show nectin-1, PSD-95 and synaptophysin immunostaining separately and overlaid in which regions of colocalization that appear white in the superimposed image. One synaptic complex is represented at high magnification in each insert. In FIG. 1C, neurons are also triple stained with nectin-1 (red), NMDA receptor 1 (NR1, green) and synaptophysin (blue). Colocalization between nectin-1 and NR1 indicates that nectin-1 localizes to glutamatergic synapses. In FIG. 1D, the subcellular localization of N-cadherin (green) is shown. N-cadherin is a cell adhesion molecule known to localize to excitatory synapses. As shown by FIG. 1E, colocalization of nectin-1 (red) and N-cadherin (green) indicates that nectin-1 localizes to the excitatory synapses and associates with N-cadherin at synapses.

Protein lysates of primary hippocampal neurons were immunoblotted with polyclonal anti-nectin-1 antibody generated against the C-terminus identified three bands of 98, 68 and 28 kDa each (FIG. 1A). The 98 kDa protein is the glycosylated mature form of nectin-1 because treatment with Endo F, but not Endo H, shifts the molecular weight of 98 kDA to 68 kDA. A 28 kDA band is the C-terminal fragment of nectin-1 produced after shedding (FIG. 1A). An analysis of the subcellular distribution of nectin-1 in primary rat hippocampal cultures showed that 95% of clusters labeled with either synaptophysin or PSD-95 were nectin-1 positive (FIG. 1B). Similar localization of nectin-1 was also observed with NMDA receptor 1 (FIG. 1C). Thus, nectin-1 appears to be concentrated at a population of asymmetric, excitatory synapses. Nectin-1 was extensively co-localized with N-cadherin at synapses indicating that they may coordinately regulate synapse formation (FIGS. 1D and 1E).

### Example 2

### The Ectodomain of Nectin-1 Undergoes Shedding

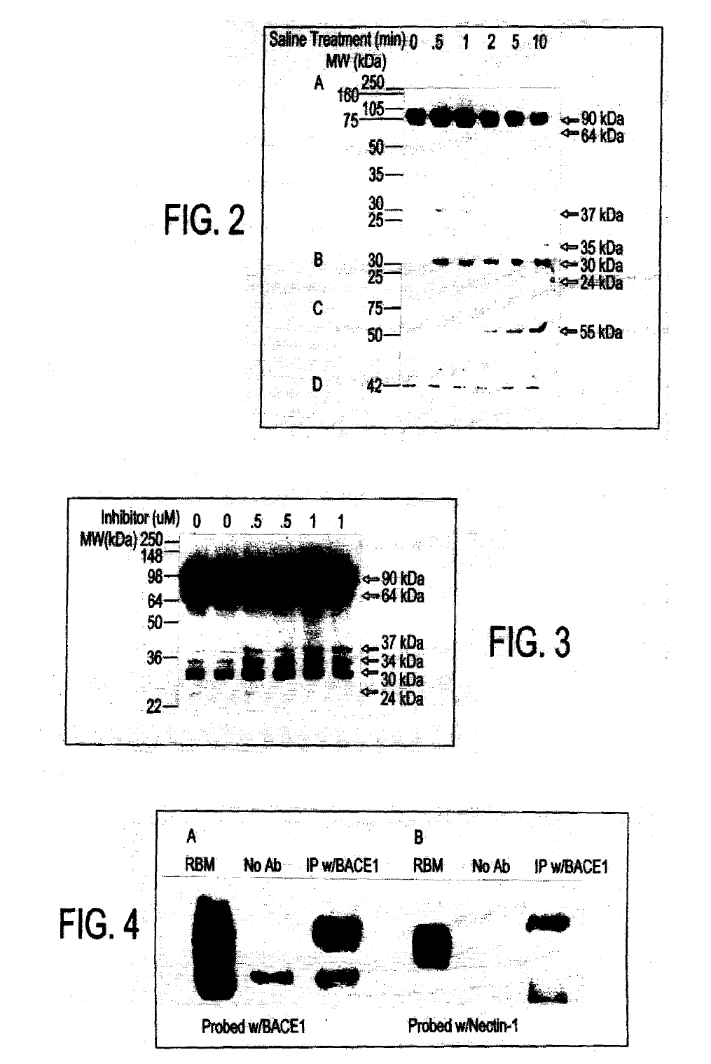

FIG. 2 shows a Western blot of the shedding of nectin-1. Neurons at 21 DIV were treated with physiological saline for 0, 0.5, 1, 2, 5, and 10 minutes, followed by collection of media and cell lysates and analysis on a 10% SDS-PAGE gel. The membranes from cell lysates (FIG. 2A) and media (FIG. 2C) were probed with polyclonal nectin-1 antibody and ectodomain specific monoclonal nectin-1 antibody, respectively. The membrane from FIG. 1A was stripped and treated with calf intestinal phosphatase (CIP) for 30 min. at 37° C. and reprobed with polyclonal nectin-1 antibody (FIG. 2B). The membrane from the cell lysate was also probed with β-actin antibody (FIG. 2D).

The ectodomain of nectin-1 is cleaved and released from Chinese hamster ovary (CHO) cells and 5-day old mouse cortical neurons in a soluble form. It was discovered that nectin-1 shedding also occurs in rat hippocampal neurons by short term treatment with physiological saline. The resultant media and whole cells lysates were analyzed by Western blot. A soluble form of 55 kDa was detected using an ectodomain specific nectin-1 antibody. The intensity of the 55 kDa band increased in a time dependent manner (FIG. 2C), whereas the amount of mature nectin-1 gradually decreased (FIG. 2A). Similar results were obtained with 55 mM KCI treatment, indicating that ectodomain shedding of nectin-1 may be activity dependent. Interestingly, a 30 kDa band that corresponds to the C-terminal fragment, was also detected in cell lysates, and declined in intensity over time (FIG. 2A). We suspected that this fragment may be phosphorylated, as it contains predicted phosphorylation sites at 5503 and 5511. A Western blot was treated with CIP to remove phosphate groups and then reprobed with nectin-1 antibody, resulting in a predominant 30 kDA band. CIP treatment also revealed two new bands in the saline treated samples, at 35 and 24 kDa. We propose that these two proteins are also derived from nectin-1 by additional enzymatic processes. Equal loading of samples was shown by beta-actin immunoblot (FIG. 2D). The saline treatment data indicates that shedding of nectin-1 is a multi-step process and may be regulated by phosphorylation.

### Example 3

### Three C-Terminal Nectin-1 Derived Fragments Accumulate in Hippocampal Neurons

Hippocampal neurons were plated at a density of 1.3×10⁵ cells. The neurons at 21 DIV were transduced with an adenovirus vector expressing a C-terminal flag tagged nectin-1 for 24 hrs at a MOI of 50. Neurons were treated without (FIG. 3, lanes 1 and 2) or with 0.5 μM (FIG. 3, lanes 3 and 4) and 1 μM γ-secretase inhibitor X (FIG. 3, lanes 5 and 6) for 24 hrs. These cells were harvested in reducing sample buffer and analyzed on a 10% SDS-PAGE gel.

Technical difficulties preclude detection of endogenous C-terminal fragments, particularly the 35 and 24 kDa forms due to low expression levels in neurons. To circumvent this, C-terminal Flag tagged nectin-1 was over expressed in neurons as described above. The Western blot exhibits six distinct molecular weight bands, 90, 64, 37, 34, 30 and 24 kDa bands. The presence of four small fragments likely indicates that there are alternative cleavage sites revealed by overexpression. Interestingly, when transduced neurons were treated with 0.5 and 1 μM γ-secretase inhibitor X, the density of three molecular bands, 37, 34 and 30 kDa, increased in the presence of γ-secretase inhibitor while the density of the 24 kDa band was reduced (FIG. 3). This suggests that the 24 kDa band is a product of γ-secretase while the others are putative substrates. These data indicate that processing of nectin-1 occurs by multiple endoproteolytic steps, one of which is catalyzed by γ-secretase.

### Example 4

### Association of Nectin-1 and BACE1 in Rat Brain

To determine if nectin-1 and BACE1 associate in rat brain, immunoprecipitation experiments were performed using a BACE1 specific antibody. Detergent lysates were prepared from rat brain under conditions that conserve protein-protein interactions. Immunoprecipitation products were subjected to immunoblot analyses to assay for the presence of nectin-1 and BACE1 (FIG. 4). Crude rat brain (lane 1), a no antibody control (lane 2) and the BACE1 immunoprecipitation product (lane 3) were all run on a 10% SDS-PAGE gel. Samples were transferred to nitrocellulose, and probed with an anti-BACE1 monoclonal antibody and detected by ECL/autoradiography using the appropriate HRP-conjugated secondary antibody (FIG. 4A). The membrane was then stripped and re-probed with anti-nectin-1 antibody and detected by ECL/autoradiography using the appropriate HRP-conjugated secondary antibody (FIG. 4B). In the second blot, the 98 kDa nectin-1 band was detected (FIG. 4B), indicating that nectin-1 associates with BACE1 in brain.

### Example 5

### BACE1 Localizes to Synapses in Cultured Hippocampal Neurons

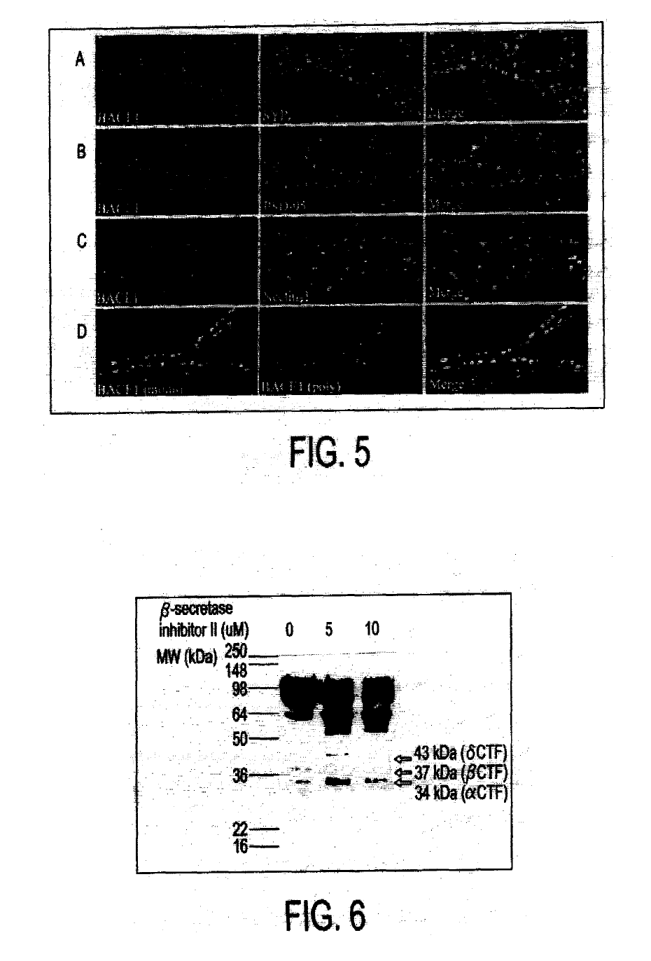

The subcellular distribution of BACE1 in hippocampal neurons was examined. Neurons at 28 DIV were methanol fixed and labeled with BACE1 (red) and two synaptic markers, synaptophysin (green) and PSD-95 (green) (FIGS. 5A and B). Quantification indicates that 98% of synaptophysin or PSD-95 puncta were associated with BACE1. Neurons were also co-stained for BACE1 (red) and nectin-1 (green). More than 95% nectin-1 puncta were near or completely co-localized with BACE1 (FIG. 5C). To confirm the specificity of the antibody, neurons were double stained with two different BACE1 antibodies raised in different species against different epitopes. The two stains superimposed completely (FIG. 5D). These data indicate that BACE1 is a synaptic molecule.

### Example 6

### β-Secretase Inhibitor Blocks the Formation of 37 kDa CTF

A pharmalogical approach was used to detect which CTF is the product of BACE1. As expression of endogenous nectin-1 CTFs are difficult to detect, HEK 293 cells were used to facilitate the analysis. The cells were transfected with wild type nectin-1 and treated with 0, 5, and 10 μM β-secretase inhibitor. Cells were harvested and lysed 24 hr. post transfection and fractionated by 10% SDS-PAGE. Samples were transferred to nitrocellulose, and the blot was probed with anti-BACE1 monoclonal antibody. Overexpression of nectin-1 generated two CTF bands, at 37 and 34 kDa (FIG. 6). Treatment of the transfected cells with β-secretase inhibitor abolished the 37 kDa band and generated a 43 kDa CTF band. These data suggest that the 37 kDa CTF is likely a product of BACE1 cleavage and that an alternative cleavage site, leading to formation of the 43 kDa band, exists upstream of the BACE1 cleavage site.

### Example 7

### BACE1 Interacts with the Ectodomain of Nectin-1

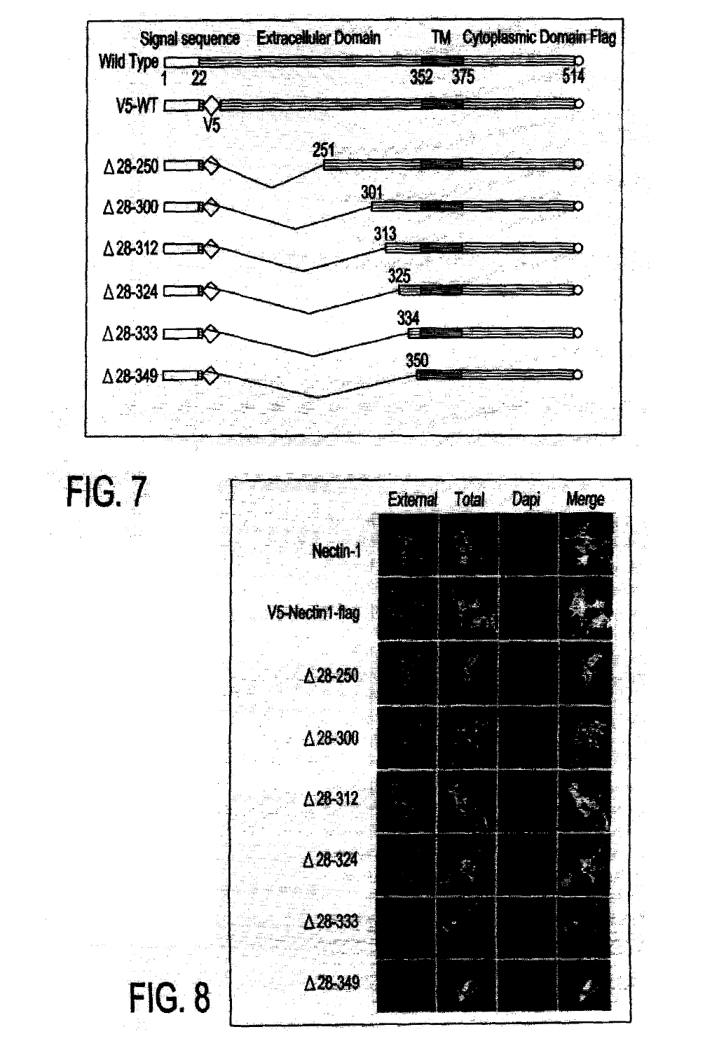

To define the minimal ectodomain region harboring cleavage sites, a molecular biology approach was used. A panel of progressively truncated mutants (FIG. 7) was generated. As depicted in FIG. 7, six external domain truncation mutants, deleted from 250 to 349 amino acids, were generated. In each of these constructs, the first and second Ig loops were completely deleted, whereas the third Ig-like loop was progressively shortened. To facilitate analysis of the mutants, a V5 epitope was appended after the signal sequence and a flag tag was appended at the c-terminus.

The constructs were first tested as to whether each was expressed on the cell surface as this is where shedding occurs. HEK 293 cells were transiently transfected with wild type and epitope tagged full-length nectin-1 and the truncation mutants. Cell surface expression was determined using a V5 antibody in the absence of detergent permeabilization. Total protein expression was then examined with cytoplasmic specific nectin-1 antibody after cell membrane permeabilization with 0.1% triton X-100. Double-tagged full-length nectin-1 was detected on the cell surface and expression levels were similar to that of wild type nectin-1. This indicates that the V5 and flag epitope tags did not disrupt cell surface expression. Surface expression of four truncation mutants, Δ28-250, Δ28-300, Δ28-312 and Δ28-324 were well detected with anti-V5 antibody (FIG. 8). Two truncations, Δ28-333 and Δ28-349 were not detected although they exhibited immunostaining with cytoplasmic antibody (FIG. 8). It is speculated that these two constructs have very short extracellular domains and, therefore, their V5 epitope tags may be buried and inaccessible to the antibody.

### Example 8

### Identification of the Minimal Domain Containing β Cleavage Site

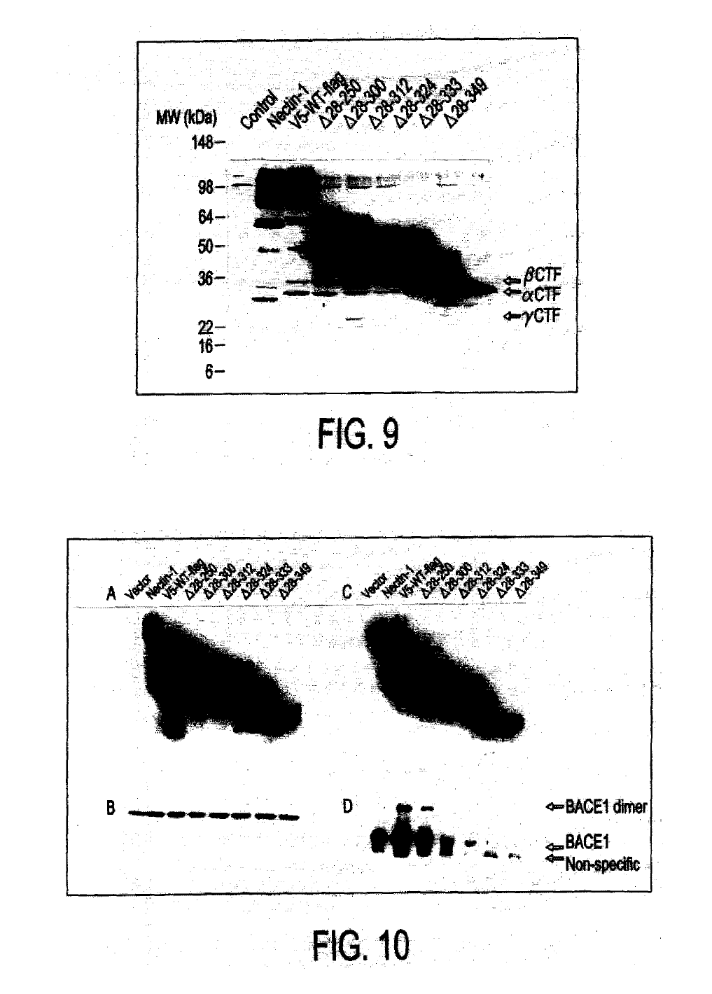

To define the minimal region that may contain the β cleavage site within the nectin-1 ectodomain, proteins from the truncated mutants were analyzed by Western blotting. Each construct was expressed in HEK 293 cells and harvested 24 hr. post transfection. Lysates were made in reducing sample buffer and analyzed on a 6 to 20% Tris-Glycine gradient gel. Samples were transferred to nitrocellulose and the blot was probed with anti-nectin-1 antibody. In negative controls, faint endogenous nectin-1 was detected but CTF bands were seen (FIG. 9). In cells transfected with wild type nectin-1, two major CTF bands (α, β) were detected without a band corresponding to γ CTF. The tagged, full length nectin-1 construct also exhibits bands corresponding to α and β CTFs, only shifted to a larger size due to the presence of the V5 and flag tags. The presence of both of these bands from the full length construct demonstrates that the tags not disrupt ectodomain cleavage. The Δ28-250, Δ28-300 and Δ28-312 constructs all exhibited the same CTF banding pattern as full-length nectin-1, indicating that the β cleavage site is downstream of residue 312 (FIG. 9). A smaller band corresponding to β CTF was exhibited by the Δ28-324 construct and no β CTF band was seen for the two remaining constructs. These data indicate that the β cleavage site of nectin-1 is located between amino acids 312 and 324.

### Example 9

### Identification of the BACE1 Interacting Domain within the Nectin-1 Ectodomain

To define the minimal region within the ectodomain of nectin-1 required for BACE1 interaction, immunoprecipitations between the nectin-1 truncation mutants and BACE1 were performed. BACE1 and full length or truncated nectin-1 were co-expressed in HEK 293 cells and harvested 48 hr. after transfection. To confirm to the efficiencies of the co-transfections, equal volumes of cell lysate were loaded on SDS-PAGE gels and analyzed by Western blot. Transfection efficiency was similar for nectin-1 and its derivates (FIG. 10A) and BACE1 (FIG. 10B). Immunoprecipitation was performed with a nectin-1 cytoplasmic specific antibody and precipitates were analyzed on two separate 10% SDS-PAGE. Western blots were probed with anti-nectin-1 (FIG. 10C) or anti-BACE1 (FIG. 10D) antibodies. The nectin-1 antibody directly immunoprecipitated the respective nectin-1 polypeptides and processed CTFs (FIG. 10C). Furthermore, BACE1, both monomer and dimer, was immunoprecipitated by the nectin-1 antibody, confirming the association of BACE1 with nectin-1. Two truncation mutants Δ28-250 and Δ28-300, interacted with BACE1 very strongly. Association with BACE1 was also detected with Δ28-312, Δ28-324 and Δ28-333, although the interaction became progressively weaker as a larger portion of the ectodomain was deleted. The final truncation mutant Δ28-349, did not associate with BACE1, indicating that the BACE1 interacting domain is located between amino acids 301 and 333 (FIG. 10D).

### Example 10

### Point Mutants Identify Three Key Amino Acids Important for Nectin-1 Processing

Site-directed alanine scanning mutagenesis was utilized to identify residue that are necessary for either cleavage or interaction with BACE1. Every amino acid from residues 301 to 334 (the putative BACE1 interaction region) was mutated to alanine, with the exception of residues 308 and 315, which were mutated to leucine. Each mutant polypeptide was expressed in HEK 293 cells. Cells were lysed in reducing sample buffer, analyzed by 10% SDS-PAGE, transferred to nitrocellulose. The Western blots were probed with anti-nectin-1 antibody. Mutation of residues 310, 311 and 323 substantially reduced a (34 kDa) and 13 (37 kDa) CTF bands and causes accumulation of 50 to 60 kDa intermediates (FIG. 11). Mutation of residues 303, 313, and 315 also causes accumulation of intermediates but still produces α and β CTF bands. The remaining mutants produced CTF bands identical to those of wild type nectin-1 (FIG. 11). These data indicate that three residues, threonine 310, tyrosine 311 and glycine 323 may play an important role in β cleavage or interaction with BACE1.

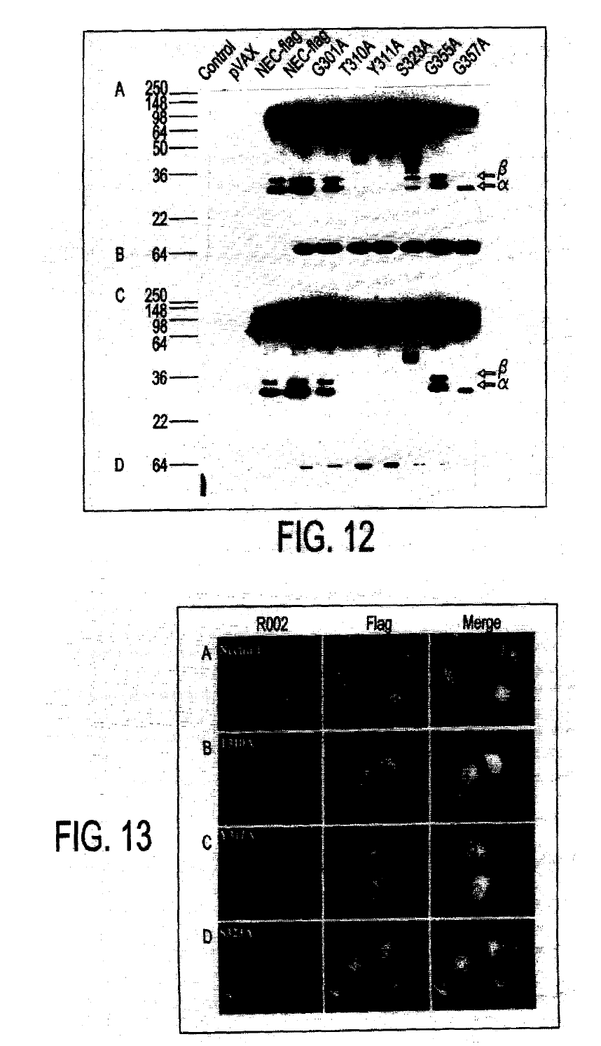

To express how these point mutations affect the association of nectin-1 with BACE1, physical association of the mutants was examined by immunoprecipitation. We co-expressed BACE1 and nectin-1 or one of the cleavage refractory point mutants in HEK 293 cells. Cells were harvested and lysed in lysis buffer 48 hr. after transfection. To confirm the co-transfection efficiency of nectin-1 or it derivatives and BACE1, equal volumes of cell lysates were analyzed by 10% SDS-PAGE followed by Western blotting. Transfection efficiencies for nectin-1 and derivatives and BACE1 were similar in each sample (FIG. 12A—nectin-1, FIG. 12B—BACE1). Co-expression of nectin-1 with BACE1 increased the amount of CTF bands compared to nectin-1 alone (FIG. 12A, lanes 3 and 4). Co-expression with BACE1 substantially increased production of CTF bands for the nectin-1 polypeptide mutant S323A and somewhat less effectively increased production in the other two mutants, T310A and Y311A (FIG. 12A).

Immunoprecipitation was performed using a C-terminal specific nectin-1 antibody. Immunoprecipitation showed that nectin-1 and its processed CTFs were detected in nectin-1 complexes (FIG. 12C). The intensities of nectin-1 CTF bands were much stronger when nectin-1 was co-expressed with BACE1. BACE1 was also detected in nectin-1 complexes (FIG. 12D). Surprisingly, the T310A and Y311A mutant polypeptides precipitated BACE1 bands with an intensity much greater than that precipitated by wild type nectin-1. This suggests that these mutants bound tightly to BACE1, and, as their cleavage was impaired, that they continue to occupy the enzyme's active site. These data also explain why these mutations block the generation of α and γ CTFs, as these mutants do not appear to dissociate from BACE1. S323A is also able to co-precipitate BACE1, however, the interaction appears to be similar to that of wild type. All other mutants, including those that interfere with α CTF band formation (G355A and G357A), and mutants with no known effect (G301A) exhibit an association with BACE1 similar to that of wild type nectin-1.

### Example 11

### Localization of Nectin-1 and its Mutants in COS-7 Cells

To examine the cellular localization of nectin-1 and point mutants of the BACE1 interacting domain, immunocytochemistry was performed. COS-7 cells transfected with flag-tagged nectin-1 or nectin-1 mutants were fixed with 3% PFA and incubated with mixtures of anti-nectin-1 (red) and flag tag (green) antibodies, followed by fluorescently labeled species-specific secondary antibodies. Expression of wild type nectin-1 was readily detected, with little evidence of intracellular accumulation (FIG. 13A). There was clear localization of nectin-1 to regions of contact, particularly when adjacent cell were also transfected (FIG. 13A). This localization to cell-cell contact regions demonstrates that nectin-1 mediates cell-cell adhesion. Mutants T310A and Y311A showed cellular localization patterns distinguishable from their wild-type counterparts (FIGS. 13B and C, respectively). Both constructs exhibited intracellular accumulation and no localization to sites of cell-cell contract, even though they were present on the cell surface (FIGS. 13B and C, respectively). Mutant S323A also accumulated intracellularly but did localize to cell-cell contact sites. However, a significant fraction of the protein was distributed over the rest of the cell surface (FIG. 13D). This indicates that mutants T310A and Y311A lost their capacity to engage in homotypic trans interactions whereas S323A retained the ability to participate in trans-dimerization. All other point mutants were indistinguishable from wild type with respect to cellular localization.

### Example 12

### Co-Expression of Nectin-1 or Nectin-1 Mutants with BACE1

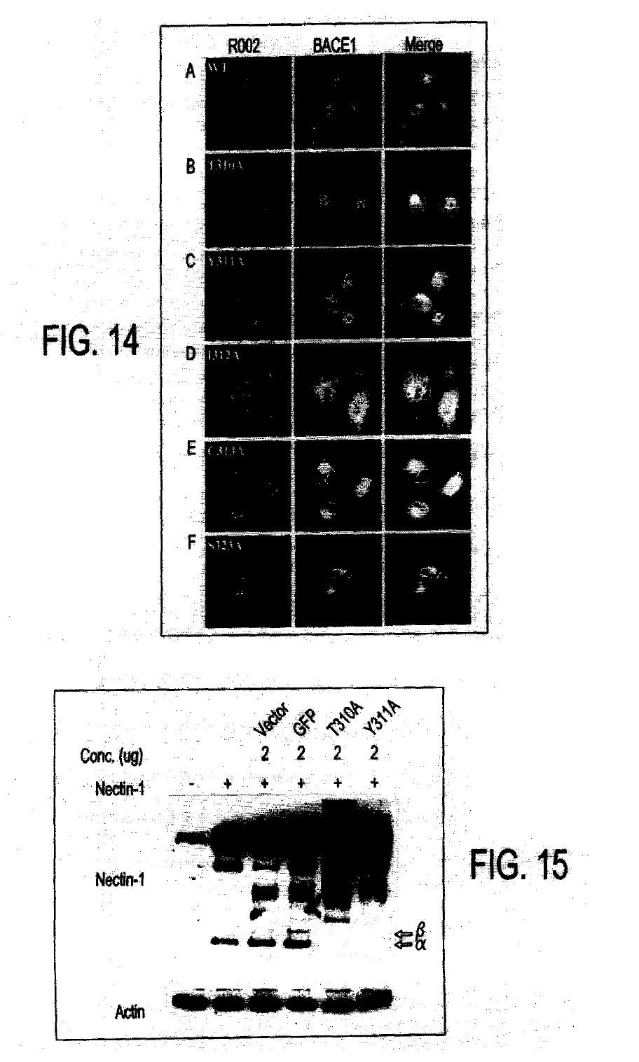

To determine whether BACE1 expression has an effect on the subcellular localization of nectin-1 and nectin-1 mutants, COS-7 cells were co-transfected with various forms of nectin-1 and a His6-tagged BACE1 construct. The cells were fixed 48 hr. after transfection and incubated with mixtures of anti-nectin-1 (red) and anti-his tag (green) antibodies followed by fluorescently labeled species-specific secondary antibodies. Cells co-expressing wild type nectin-1 and BACE1 showed virtually no co-localization at sites of cell-cell contact but did show co-localization within the ER and Golgi structures (FIG. 14A). Nectin-1 mutants T310A, Y311A and C313A showed complete co-localization with BACE1 within intracellular regions, but, interestingly, these mutants did not accumulate at sites of cell-cell contact (FIGS. 14B, C and E, respectively). Mutant S323A also completely co-localized with BACE1 and no longer accumulated at cell-cell contact sites (FIG. 14F). All other point mutants were indistinguishable from wildtype. A representative example of the other point mutants is shown in FIG. 14D. While co-localization can not be considered formal proof of a protein-protein interaction, these observations suggest that interactions of the nectin-1 mutants form with BACE1 in intracellular compartments.

### Example 13

### T310A and Y311A Trans-Dominantly Interfere with Processing of Wild Type Nectin-1

Mutants T310A and Y311A were tested to see if they would interfere with the processing of wild type nectin-1. HEK 293 cells were co-transfected with 0.5 μg of a plasmid containing wild-type nectin-1 and with a plasmid containing no insert (control), green fluorescent protein (GFP), nectin-1 T310A or nectin-1 Y311A. Co transfection efficiency was approximately 85%. Cells were harvested 24 hr. post-transfection and samples were analyzed on a 12% SDS-PAGE gel. Western blots were probed with anti-nectin-1 antibody or β-actin antibody to demonstrate equal loading of samples. Co-transfection with vector alone or GFP had no effect on nectin-1 processing. Co-transfection of nectin-1 T310A and Y311A substantially reduced the production of CTF bands (FIG. 15). The residual CTF bands detected in the T310A and Y311A samples are likely due to the 15% of the cell population that received only the wild type nectin-1 construct. These data indicate that nectin-1 T310 and Y311A can trans-dominantly interfere with processing of nectin-1.

What is claimed is:

1. A method for diagnosing Alzheimer's disease or a predilection to Alzheimer's disease in a subject comprising,
obtaining a sample from the subject; and
analyzing proteins present in the sample for the presence of fragments of amino acid sequence SEQ ID NO:1, wherein the presence of fragments of amino acid sequence SEQ ID NO:1 indicates a diagnosis of Alzheimer's disease or a predilection to Alzheimer's disease.

2. The method of claim 1, wherein one or more of the fragments of SEQ ID NO:1 are the products of cleavage by α secretase.

3. The method of claim 1, wherein one or more of the fragments of SEQ ID NO:1 are the products of cleavage by γ secretase.

4. The method of claim 1, wherein one or more of the fragments of SEQ ID NO:1 are the products of cleavage by BACE1.

5. The method of claim 1, wherein one or more of the fragments of SEQ ID NO:1 are the products of cleavage by a BACE 1-like protease.

6. The method of claim 1, wherein the analysis is done by two-dimensional gel electrophoresis.

7. The method of claim 1, wherein the analysis is done by mass spectrometry.

8. The method of claim 1, wherein the analysis is done by Western blot.

9. The method of claim 1, wherein the sample is a bodily fluid.

10. The method of claim 9, wherein the bodily fluid is cerebrospinal fluid.

11. The method of claim 9, wherein the bodily fluid is blood.

12. The method of claim 9, wherein the bodily fluid is urine.

13. The method of claim 1, wherein Herpes Simplex Virus is administered to the subject before obtaining a sample from the subject.

14. A method for developing a database for diagnosing Alzheimer's disease comprising,
providing a group of subjects, wherein some of the subjects exhibit symptoms of Alzheimer's disease;
obtaining samples from the subjects;
analyzing proteins present in the sample for the presence of fragments of amino acid sequence SEQ ID NO:1; and
developing an entry in the database for each subject which correlates the symptoms exhibited by the subject with the fragments of amino acid sequence SEQ ID NO:1 present in the sample from the subject.

15. The method of claim 14, wherein one or more of the fragments of SEQ ID NO:1 are the products of cleavage by α secretase.

16. The method of claim 14, wherein one or more of the fragments of SEQ ID NO:1 are the products of cleavage by γ secretase.

17. The method of claim 14, wherein one or more of the fragments of SEQ ID NO:1 are the products of cleavage by BACE1.

18. The method of claim 14, wherein one or more of the fragments of SEQ ID NO:1 are the products of cleavage by a BACE1-like protease.

19. A method for diagnosing Alzheimer's disease or a predilection to Alzheimer's disease in a subject, using the database of claim 14, comprising:
obtaining a sample from the subject;
analyzing proteins present in the sample for the presence of fragments of amino acid sequence SEQ ID NO:1; and
comparing the fragments of SEQ ID NO:1 in the sample of the subject with the fragments of SEQ ID NO:1 of the entries in the database, wherein, if the fragments of SEQ ID NO:1 are similar to the fragments of one or more entries in the database that correlate with Alzheimer's disease, the subject is diagnosed as having Alzheimer's disease or a predilection to Alzheimer's disease.

20. A nucleic acid sequence encoding a polypeptide comprising an amino acid sequence selected from the group consisting of: SEQ ID NO:3, SEQ ID NO:4, SEQ ID NO:5, SEQ ID NO:6, SEQ ID NO:7, SEQ ID NO:8, SEQ ID NO:9, or SEQ ID NO:10.

21. A substantially purified polypeptide comprising an amino acid sequence selected from the group consisting of: SEQ ID NO:3, SEQ ID NO:4, SEQ ID NO:5, SEQ ID NO:6, SEQ ID NO:7, SEQ ID NO:8, SEQ ID NO:9, or SEQ ID NO:10.
